(19) United States
(12) Patent Application Publication
(10) Pub. No.: US 2014/0079807 A1
(43) Pub. Date: Mar. 20, 2014

(54) ANTIMICROBIAL ACTION OF COPPER IN GLASS

(71) Applicants: Nicholas Francis Borrelli, Elmira, NY (US); Odessa Natalie Petzold, Elmira, NY (US); Joseph Francis Schroeder, Corning, NY (US); Thomas Philip Seward, Elmira, NY (US); Florence Verrier, Corning, NY (US); Ying Wei, Painted Post, NY (US)

(72) Inventors: Nicholas Francis Borrelli, Elmira, NY (US); Odessa Natalie Petzold, Elmira, NY (US); Joseph Francis Schroeder, Corning, NY (US); Thomas Philip Seward, Elmira, NY (US); Florence Verrier, Corning, NY (US); Ying Wei, Painted Post, NY (US)

(73) Assignee: Corning

(21) Appl. No.: 14/007,810

(22) PCT Filed: Mar. 27, 2012
(86) PCT No.: PCT/US12/30704

§ 371 Date: Sep. 26, 2013

Related U.S. Application Data
(60) Provisional application No. 61/468,153, filed on Mar. 28, 2011.

(51) Int. Cl. A61K 33/34; C03C 21/00; A61K 33/30; A61K 33/22; A61K 33/08; C03C 23/00; A61K 33/00

(52) U.S. Cl. CPC A61K 33/34; C03C 23/007; C03C 21/002; A61K 33/00; A61K 33/22; A61K 33/08; A61K 33/30
USPC 424/635; 65/32.5; 65/30.14; 428/409; 428/433; 424/724; 424/657; 424/682; 424/641; 424/722

(57) ABSTRACT

The disclosure is directed to glass compositions that incorporate copper into an otherwise homogeneous glass and to a method for making such glass. This incorporation of the copper into the glass composition imparts significant antimicrobial activity to the glass. A method of making a copper-containing glass article comprises: batching a glass batch comprising: 40-85 SiO₂; 10-40 B₂O₃; 1-19 Al₂O₃; 0.1-20 CuO or a selected salt of Cu that is convertible into CuO during melting; 0-20 M₂O, wherein M is Li, Na, K, or combinations thereof; 0-25 RO, wherein R is Ca, Sr, Mg, or combinations thereof; and 0-20 ZnO. melting the batch to form a melted glass; and forming the melted glass to form the copper-containing glass article having antimicrobial properties.

### CROSS-REFERENCE TO RELATED APPLICATIONS

This application claims the benefit of priority under 35 U.S.C. §119 of U.S. Provisional Application Ser. No. 61/468,153 filed on Mar. 28, 2011 the content of which is relied upon and incorporated herein by reference in its entirety.

### BACKGROUND

1. Field

This disclosure is directed to the production of glass whose surfaces have antimicrobial activity, and in particular to glass surfaces containing copper. The disclosure ifs further directed to a method of making such copper-containing glass and articles from the glass.

2. Technical Background

There is patent and otherwise published literature dealing with antimicrobial action, for example, the antibacterial action of silver both in the ionic form and the nanoparticle form. While antibacterial activity is desirable for many reasons in different applications, a clear distinction is to be made between antibacterial activity and antiviral activity. The distinction is made on the grounds that the mechanism by which metals such as silver alter or kill bacteria may not be the same as the mechanism that metals kill viruses. Moreover, with reference to antiviral activity, the mention of other metals or metal ions other than silver is rare. Articles referring to the antiviral activity of copper, copper alloys and copper ion include J. O. Noyce et al, “Inactivation of influenza A virus on copper versus stainless steel surfaces”. Appl. Environ. Microbiol. Vol. 73 (2007) pages 2748-2750; J. L. Sagripanti et al, “Cupric and ferric ions inactivate HIV,” AIDS Res Hum Retroviruses Vol. 12 (1966), pages 333-337; and J. L. Sagripanti, “Mechanism of copper-mediated inactivation of herpes simplex virus,” Antimicrob. Agents Chemother., Vol. 41 (1997), pages 12-817. These articles discuss antiviral properties of copper; more specifically, the antiviral action in a Cu⁺² solution and a metallic copper surface.

There is a need for glasses with antimicrobial properties in applications such as medical applications wherein surfaces come in contact with humans.

### SUMMARY

Embodiments are directed to glass articles that incorporate copper ions, copper metal, and/or colloidal copper such as copper nanoparticles into an otherwise homogeneous glass and to a method for making such glass articles. This incorporation of the copper into the glass articles promotes significant antimicrobial activity such as antibacterial and/or antiviral activity. One advantage of embodiments described herein is a strong and smooth antiviral glass surface that is useful for a variety of applications where this property is either desirable or necessary. The antimicrobial property is integral to the glass, and is not a coating applied to the surface that will not either wear off or be removed. Applications in which the antiviral glass can be used include medical, healthcare, laboratory shelving and surfaces, and appliance surfaces where antimicrobial function would provide benefit.

One embodiment is a glass article comprising copper selected from the group consisting of Cu ions, metallic copper, colloidal copper, and combinations thereof dispersed throughout the glass and at a surface of the glass; and the glass having antimicrobial properties.

Another embodiment is a method of making a copper-containing glass article having antimicrobial properties, the method comprises:
batching a glass batch comprising:
   40-85 SiO₂;
   10-40 B₂O₃;
   1-19 Al₂O₃;
   0.1-20 CuO or a selected salt of Cu that is convertible into CuO during melting;
   0-20 M₂O, wherein M is Li, Na, K, or combinations thereof;
   0-25 RO, wherein R is Ca, Sr, Mg, or combinations thereof; and
   0-20 ZnO.
melting the batch to form a melted glass; and
forming the melted glass to form the copper-containing glass article having antimicrobial properties.

Additional features and advantages of the invention will be set forth in the detailed description which follows, and in part will be readily apparent to those skilled in the art from the description or recognized by practicing the invention as described in the written description and claims hereof, as well as the appended drawings.

It is to be understood that both the foregoing general description and the following detailed description are merely exemplary of the invention, and are intended to provide an overview or framework for understanding the nature and character of the invention as it is claimed.

The accompanying drawings are included to provide a further understanding of the invention, and are incorporated in and constitute a part of this specification. The drawings illustrate one or more embodiment(s) of the invention and together with the description serve to explain the principles and operation of the invention.

### BRIEF DESCRIPTION OF THE DRAWINGS

The invention can be understood from the following detailed description either alone or together with the accompanying drawing figures.

FIG. 1 is a SEM microphotograph of a glass having a high density of Cu-nanoparticles at the surface and extending into the glass for an approximate distance of 5 μm according to one embodiment.

### DETAILED DESCRIPTION

Reference will now be made in detail to various embodiments of the invention, examples of which are illustrated in the accompanying drawings. Wherever possible, the same reference numbers will be used throughout the drawings to refer to the same or like parts.

As used herein the term “antimicrobial,” means an agent or material, or a surface containing the agent or material that will kill or inhibit the growth of at least two different types of microbes: bacteria, viruses and fungi. The term as used herein does not mean it will kill or inhibit the growth of all species microbes within such families, but that it will kill or inhibit the growth or one or more species of microbes from such families. When an agent is described as being “antibacterial, or “antiviral” or “antifungal,” it means that the agent will kill or inhibit the growth of bacteria, viruses or fungi, respectively.

As used herein the term “Log Reduction” or “LR” means −Log(Ca/C₀), where Cₐ=the colony form unit (CFU) number of the antimicrobial surface containing Cu nanoparticles and C₀=the colony form unit (CFU) of the control glass surface that does not contain Cu nanoparticles. That is:

LR=−Log(Cₐ/C₀),

As an example, a Log Reduction of 3=99.9% of the bacteria or virus killed and a Log Reduction of 5=99.999% of bacteria or virus killed.

The test method used for determining antibacterial properties of a copper-containing glass was a modified version of the JISZ-2801: 2000 method, which is a Japanese Industrial Standard that was developed to measure the antibacterial activity of copper-containing glass. The antibacterial activity is measured by quantitatively by determining the survival of bacteria cells that have been held in intimate contact with a surface thought to be antibacterial and incubated for 24 hours at 35° C. After the time period has elapsed the cells are counted and compared to a non-treated surface. The test was modified in that for the incubation period was changed to 6 hours at 37° C. After 6 hours the samples were removed from the incubator and the entire testing surface was thoroughly washed with PBS to ensure that all bacteria were removed. The cells and the PBS wash were then transferred to a broth agar plate for overnight culture. After a period of 16-24 hours the bacterial colonies on the agar plate were counted. 150 μl of bacterial suspension of concentration 1×10⁶ cells/ml was added to the sample plates which can be either a copper-containing glass plate or a control (no copper) plate, covering the plates having a bacterial suspension thereon with the PARAFILM® resulting in PARAFILM® covered plates, and thereafter incubating the bacteria at 37° C. for 6 hours as indicated by, and lastly counting the colonies. The samples were tested using E. coli (gram negative) bacteria.

One embodiment is a glass article comprising copper selected from the group consisting of Cu ions, metallic copper, colloidal copper, and combinations thereof dispersed throughout the glass and at a surface of the glass; and the glass having antimicrobial properties. The copper (whether as the Cu⁺¹, Cu⁺², in the reduced state as a Cu nanoparticle) can be at the surface of the glass, a portion of the copper can be embedded or partially embedded in the glass, and/or the copper in any form can be dispersed throughout the glass article, including the surface. The article and the glass can be phosphorus free, for example, free from any intentionally added phosphorus.

In one embodiment, the copper is in a reduced state; and the glass article has antimicrobial properties, for example, antiviral and/or antibacterial. In one embodiment, the copper is in a reduced state; and the glass article has antiviral properties. In one embodiment, the copper is in a reduced state; and the glass article has antibacterial properties. The reduced copper can be at a depth of in the range of from 2 μm to 3 μm from the surface of the glass. In one embodiment, in the reduced copper case, the copper nanoparticles are on the surface and extending to a depth of in the range of from 2 μm to 3 μm from the surface of the glass. In one embodiment, the copper is robustly and tenaciously adhered to the surface, that is, the copper on the surface cannot be removed by wiping or cleaning. The article can have a log reduction ≧1, for example, ≧2, for example, ≧3, for example, ≧4.

In one embodiment, the glass has antibacterial properties. The article can have a log reduction ≧1, for example, ≧2, for example, ≧3, for example, ≧4.

The glass can be a strengthened glass, for example, an ion-exchanged glass.

The glass as batched can comprise 0.1 mole %-20 mole % copper, for example, 1-16 mole %, for example, 5-16 mole %, for example, 5-15 mole %.

In one embodiment the glass as batched consists essentially of SiO₂=47±2 mole %, Al₂O₃=9±1-1.5 mole %, B₂O₃=27±3 mole %, 7-16±1.5 mole % for ZnO, and Cu being 0.5-10±0.2-1.5 mole % as the copper content increases.

The glass as batched can comprise 10 mole %-40 mole % B₂O₃. The glass as batched can comprise a B₂O₃/Al₂O₃ ratio greater than 1, for example, greater than 2, for example, greater than 3. The glass as batched, in one embodiment, comprises in mole percent:
40-85 SiO₂;
10-40 B₂O₃;
1-19 Al₂O₃;
0.1-20 CuO;
0-20M₂O, wherein M is Li, Na, K, or combinations thereof
0-25 RO, wherein R is Ca, Sr, Mg, or combinations thereof and
0-20 ZnO.

The glass as batched can be phosphorus free.

In one embodiment, the glass as batched comprises:
40-70 SiO₂;
16-31 B₂O₃;
3-15 Al₂O₃;
5-15 CuO;
0-20 M₂O, wherein M is Li, Na, K, or combinations thereof
0-25 RO, wherein R is Ca, Sr, Mg, or combinations thereof and
0-17 ZnO.

Another embodiment is a method of making a copper-containing glass article having antimicrobial properties, the method comprises:
batching a glass batch comprising:
   40-85 SiO₂;
   10-40 B₂O₃;
   1-19 Al₂O₃;
   0.1-20 CuO or a selected salt of Cu that is convertible into CuO during melting;
   0-20 M₂O, wherein M is Li, Na, K, or combinations thereof
   0-25 RO, wherein R is Ca, Sr, Mg, or combinations thereof and
   0-20 ZnO.
melting the batch to form a melted glass; and
   forming the melted glass to form the copper-containing glass article having antimicrobial properties.

The method, according to one embodiment, further comprises heating the article in a reducing atmosphere at an elevated temperature in the range of from 250° C. to 475° C. thereby reducing the copper ions, Cu⁺², in the glass as an oxide or other species, to the metal, Cu⁰. The heating can comprise heating the article for a time in the range of from 1 hour to 5 hours, for example, 2 to 5 hours. In one embodiment, the reducing atmosphere comprises hydrogen.

The method can further comprise strengthening the article after the forming. The strengthening, in one embodiment, comprises ion-exchanging alkali metal ions in the article for alkali metal ions that have a larger ionic radius.

The exemplary glass compositions can enable the incorporation of high concentrations of copper oxide into easily formed homogeneous glass batches. The examples 1-9 given in Table 1 are exemplary glass batches in mole %, and do not include all the possible compositions spanning a range of glass families, for example, borate glasses, aluminoborosilicate glasses, alkali aluminoborosilicate glasses, soda lime glass. As shown in Table 1, the copper in the glass as batched, determined as the oxide, is in the range of 0.5 mol % to 16 mol %. The compositions in Table 1 are batched compositions. Compositions as shown in Table 1 can have, for example, a variation of ±2 mole % for SiO₂, ±3 mole % for B₂O₃, ±1-1.5 mole % for Al₂O₃ and ZnO will have substantially similar activity.

TABLE 1

| Examples | 1 | 2 | 3 | 4 | 5 | 6 | 7 | 8 | 9 |
|---|---|---|---|---|---|---|---|---|---|
| SiO₂ | 47 | 47 | 47 | 47 | 47 | 47 | 47 | 47 | 47 |
| Al₂O₃ | 9 | 9 | 9 | 9 | 9 | 9 | 9 | 9 | 9 |
| B₂O₃ | 27 | 27 | 27 | 27 | 27 | 27 | 27 | 27 | 27 |
| CuO | 10 | 5 | 1 | 0.5 | 10 | 12 | 14 | 16 | 7.5 |
| ZnO | 7 | 12 | 16 | 16.5 | 7 | 5 | 3 | 1 | 9.5 |

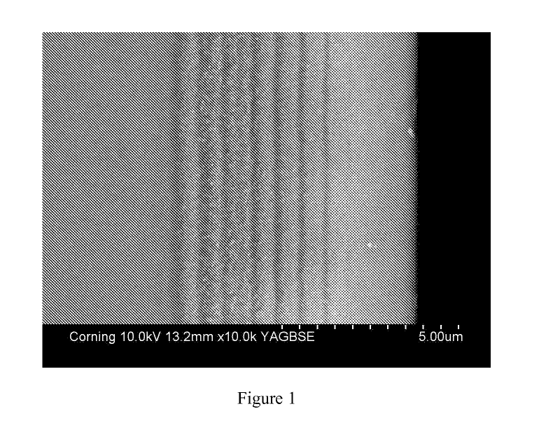

After melting the batch materials, the glass may be formed using conventional glass forming methods, for example without limitation, slot draw, fusion draw and/or the float method. The glass article can be a sheet and, in some embodiments, has a thickness in the range of 0.3 mm to 5 mm, and the length and width can be varied. In other embodiments, the glass article can be arbitrary shapes, for example, conformal to curved surfaces or in the shape of a tube and, in some embodiments, has a thickness in the range of 0.3 mm to 5 mm, and the length and width can be varied. Once a glass article is formed, it can be cut into individual articles and further treated or the entire sheet can be further treated and then cut into the articles. In either case the further treatment of the as-made Cu-containing glasses constitutes the next step in which the glass is treated in a hydrogen atmosphere at a temperature of 450° C. for 5 hours to reduce the Cu⁺¹, and/or Cu⁺² in the glass to Cu⁰. The process yields a high density of metallic copper nanoparticles at the glass's surface and extending, for example, 5 μm into the glass as shown in the SEM Micrograph of FIG. 1. In FIG. 1 the glass surface is to the right. The bright patterns are indicative of copper nanoparticles.

Glasses containing B₂O₃ have a tendency to phase separate into a borate-rich and borate-poor phase. The Cu likely goes into the borate-rich phase, therefore locally enriching the Cu concentration which can be beneficial. The addition of Al₂O₃ suppresses the phase separation tendency. The role of Zn can also play the same role. The following Tables 2 and 3 show the range of the constituents (B, Al, Zn).

Table 2 shows exemplary glass batches in mole %, examples 10-15, having Al₂O₃/B₂O₃ and SrO variations at 1% CuO.

TABLE 2

| Examples | 10 | 11 | 12 | 13 | 14 | 15 |
|---|---|---|---|---|---|---|
| SiO₂ | 47 | 47 | 47 | 47 | 47 | 47 |
| Al₂O₃ | 9 | 11 | 13 | 15 | 9 | 9 |
| B₂O₃ | 27 | 25 | 23 | 21 | 27 | 27 |
| CuO | 1 | 1 | 1 | 1 | 1 | 1 |
| ZnO | 16 | 16 | 16 | 16 | 7 | 0 |
| SrO | 0 | 0 | 0 | 0 | 9 | 16 |

Table 3 shows exemplary glass batches in mole %, examples 16-21, having Al₂O₃/B₂O₃ and SrO variations at 5% CuO.

TABLE 3

| Examples | 16 | 17 | 18 | 19 | 20 | 21 |
|---|---|---|---|---|---|---|
| SiO₂ | 47 | 47 | 47 | 47 | 47 | 47 |
| Al₂O₃ | 9 | 11 | 13 | 15 | 9 | 9 |
| B₂O₃ | 27 | 25 | 23 | 21 | 27 | 27 |
| CuO | 5 | 5 | 5 | 5 | 5 | 5 |
| ZnO | 12 | 12 | 12 | 12 | 6 | 0 |
| SrO | 0 | 0 | 0 | 0 | 9 | 16 |

### Antibacterial Testing

Antibacterial tests were carried out using cultured gram negative E. coli; DHSalpha-Invitrogen Catalog No. 18258012, Lot No. 7672225, rendered Kanamycin resistant through a transformation with PucI9 (Invitogen) plasmid). The bacteria culture was started using either LB Kan Broth (Teknova #L8145) or Typtic Soy Broth (Teknova # T1550). Approximately 2 μl of liquid bacteria suspension or a pipette tip full of bacteria were streaked from an agar plate and dispensed into a capped tube containing 2-3 ml of broth and incubated overnight at 37° C. in a shaking incubator. The next day the bacteria culture was removed from the incubator and washed twice with PBS. The optical density (OD) was measured and the cell culture was diluted to a final bacterial concentration of approximately 1×10⁶ CFU/ml. The cells were placed on the selected glass surface, antimicrobial or not antimicrobial (the control) for 6 hours at a temperature of 37° C. The buffers from each well were collected and the plates were twice washed with ice-cold PBS. For each well the buffer and wash were combined and the surface spread-plate method was used for colony counting.

### Antiviral Testing

The antiviral test procedure was carried out using a modified protocol previously described by A. Klibanov. et al, Nature Protocols (2007). Briefly, Adenovirus Type 5 was diluted to approximately 10⁶ PFU/ml in phosphate buffered saline (PBS). Adenovirus solution (10 μl) was applied to the glass slide for 2 hours at room temperature. Virus-exposed to the slides are then collected by thorough washes with PBS. Washing suspension containing the viruses were then serially diluted 2-fold with sterilized PBS and 50 μL of each dilution was used to infect HeLa cells grown as a monolayer in a 96 well microplate. After two days, viral titer was calculated by counting the number of infected HeLa cells. Virus titer reduction was calculated as previously described (Standard test method for efficacy of sanitizers recommended for inanimate Non-food contact surfaces, E1153-03, re-approved 2010). The % reduction equals:

[(number of virus surviving on the glass control−number of virus surviving on the sample glass)×100]÷number of virus surviving on the glass control.

Exemplary glasses 1, 2, 6, and 9 from Table 1 were hydrogen treated at 450° C. for 5 hours and tested for E. coli. The antibacterial results from the JIS Z 2801 test are as follows in Table 4.

TABLE 4

| | | Log |
|---|---|---|
| Example | CuO | reduction |
| 2 | 5% | log 5 |
| 9 | 7.50% | log 4 |
| 1 | 10% | log 5 |
| 6 | 12% | log 5 |

Exemplary glasses 2, 5, 6, and 7 from Table 1 were hydrogen treated at 450° C. for 5 hours and tested against adenovirus. Exemplary glasses 2, 5, 6, and 7 showed adenovirus log reduction 5 or greater.

Exemplary glasses as batched 1 and 2 in Table 1, which have been described above as having with significant antibacterial behavior, also exhibit a very potent antiviral activity, with virus titer reduction after 2 hours of exposure reaching 100% (4.5 log reduction) compared to the glass control. Interestingly, when the same glasses that were not subjected to reducing conditions and where Cu is present as a form of ions, the samples did not show significant antiviral activity. However, these same glasses showed antibacterial activity. These results indicate that a high concentration of nano-size metallic copper particles at the surface of the glass is responsible for the strong antiviral activity. Moreover, these results suggest that there is a different mode of action for these Cu glass samples when acting against bacteria versus when acting against viruses. The “kill” mechanisms are different for viruses and bacteria.

Table 5 shows exemplary glass batches in mole %, examples 22-27, having CuO added to a Pyrex®, an aluminoborosilicate glass, base glass at levels of 0.25, 0.5, 1, 2.5, and 5 mole %.

TABLE 5

| Examples | 22 | 23 | 24 | 25 | 26 | 27 |
|---|---|---|---|---|---|---|
| SiO₂ | 83.27 | 83.27 | 83.27 | 83.27 | 83.27 | 83.27 |
| Al₂O₃ | 1.21 | 1.21 | 1.21 | 1.21 | 1.21 | 1.21 |
| B₂O₃ | 11.53 | 11.53 | 11.53 | 11.53 | 11.53 | 11.53 |
| Na₂O | 3.99 | 3.99 | 3.99 | 3.99 | 3.99 | 3.99 |
| CuO | 0 | 0.25 | 0.5 | 1 | 2.5 | 5.0 |
| R-value | 0.24 | 0.24 | 0.24 | 0.24 | 0.24 | 0.24 |

Exemplary glasses were hydrogen treated at 450° C. for 5 hours. Antibacterial JIS Z 2801 test was performed using E. coli with exemplary glass 24 and 27 having a log reduction of greater than 1. Exemplary glass 27 had a log reduction of greater than 1.5. Similar results on non-reduced glasses were obtained.

Table 6 shows exemplary glass batches in mole %, examples 28-33, having CuO added to a Vycor®, an aluminoborosilicate glass batch, at levels of 0.25, 0.5, 1, 2.5 and 5 mole %.

TABLE 6

| Examples | 28 | 29 | 30 | 31 | 32 | 33 |
|---|---|---|---|---|---|---|
| SiO₂ | 64.39 | 64.39 | 64.39 | 64.39 | 64.39 | 64.39 |
| Al₂O₃ | 1.55 | 1.55 | 1.55 | 1.55 | 1.55 | 1.55 |
| B₂O₃ | 26.34 | 26.34 | 26.34 | 26.34 | 26.34 | 26.34 |
| Na₂O | 7.72 | 7.72 | 7.72 | 7.72 | 7.72 | 7.72 |
| CuO | 0 | 0.25 | 0.5 | 1 | 2.5 | 5 |
| R-value | 0.23 | 0.23 | 0.23 | 0.23 | 0.23 | 0.23 |

Exemplary glasses 29, 30, and 31 were hydrogen treated at 450° C. for 5 hours and antibacterial tested for E. coli using the JIS Z 2801 test. Exemplary glass 29 had a log reduction of 2.3 to 5, exemplary glass 30 had a log reduction of 5, and exemplary glass 31 had a log reduction of 3.5. Non-reduced glasses had similar results.

Exemplary glass 29 was hydrogen treated at 450° C. for 1 or 2 hours. In the antiviral testing using adenovirus, the log reduction was about 2.

Table 7 shows exemplary glass batches, in mole %, examples 34-39, having CuO added to a borosilicate glass batch, at levels as shown in Table 7.

TABLE 7

| Examples | 34 | 35 | 36 | 37 | 38 | 39 |
|---|---|---|---|---|---|---|
| SiO₂ | 71.94 | 71.97 | 71.61 | 71.36 | 71.33 | 70.87 |
| Al₂O₃ | 0 | 0 | 3.3 | 5.47 | 5.61 | 7.79 |
| B₂O₃ | 25.1 | 25.06 | 22.15 | 20.22 | 20.1 | 18.55 |
| Li₂O | 2.54 | 2.55 | 2.52 | 2.52 | 2.54 | 2.52 |
| K₂O | 0.42 | 0.42 | 0.42 | 0.42 | 0.42 | 0.42 |
| CuO | 5.85 | 8.65 | 5.82 | 5.81 | 8.56 | 8.51 |
| R-value | 0.12 | 0.12 | −0.02 | −0.13 | −0.13 | −0.26 |

Exemplary glass 39 was hydrogen treated at 450° C. for 5 hours and antibacterial tested for E. coli. Exemplary glass 39 had a log reduction of 5 in the JIS Z 2801 test results.

Exemplary glass 36 was hydrogen treated at 450° C. for 5 hours and antiviral tested with adenovirus with a log reduction of 5.

Table 8 shows exemplary glass batches, in mole %, examples 40-45, having CuO added to an aluminoborosilicate glass batch, at levels as shown in Table 8.

TABLE 8

| Examples | 40 | 41 | 42 | 43 | 44 | 45 |
|---|---|---|---|---|---|---|
| SiO₂ | 63.8 | 63.8 | 63.8 | 63.8 | 63.8 | 63.8 |
| B₂O₃ | 17.2 | 17.2 | 17.2 | 17.2 | 17.2 | 17.2 |
| Al₂O₃ | 6.1 | 6.1 | 6.1 | 6.1 | 6.1 | 6.1 |
| Na₂O | 1.8 | 1.8 | 1.8 | 1.8 | 1.8 | 1.8 |
| Li₂O | 4.16 | 4.16 | 4.16 | 4.16 | 4.16 | 4.16 |
| K₂O | 6.81 | 6.81 | 6.81 | 6.81 | 6.81 | 6.81 |
| CuO | 0 | 0.5 | 1 | 2.5 | 5 | 7.5 |
| SnO₂ | 0.01 | 0.01 | 0.01 | 0.01 | 0.01 | 0.01 |
| R-value | 0.39 | | | | | |
| Anneal | 485 C. | | | | | |

Exemplary glass 45 was hydrogen treated at 450° C. for 5 hours and antibacterial tested for E. coli. Exemplary glass 45 had a log reduction of 5 in the test. Non-reduced glass 45 had similar results.

Exemplary glass 45 was antiviral tested with adenovirus and had a log reduction of 2.

Table 9 shows antiviral testing results for exemplary glasses 2, 6, 7, 33, 36, and 45. Exemplary glass 7, 2, 6, 36 and 33 had extremely strong and broad antiviral activity. Morever, exemplary glass 2 killed 5 log of HSV very fast (5 min).

TABLE 9

| Example | Mole % | Antiviral activity (logreduction/time) | | | |
|---|---|---|---|---|---|
| Number | Cu | Adenovirus | HIV-1 | Influenza A | HSV |
| 45 | 7.5 | 2/2 h | 1.5/1 h | NT | NT |
| 7 | 3 | 5/2 h | NT | TBD | 3.8/1 h |
| 2 | 5 | 5/2 h | 5.32/1 h | TBD | 5/5 min |
| 6 | 10 or 12 | 5/2 h | 3.92/1 h | 2.5/30 min | 5/1 h |
| 36 | 5.8 | 5/2 h | | | |
| 33 | 5 | 4.3/2 h | | | |

In one embodiment, the glass as batched has an R-value of less than 1. The role of R-value in the borosilicate glasses may influence the antimicrobial behavior and the ability to precipitate the copper nanoparticles within the volume of the glass, as opposed to on the surface, where it can be wiped off. R-value provides an indication of the number of NBOs (non-bridging oxygens) in the glass structure.

R-value is defined as the ratio of (total alkali minus alumina)/boric oxide, in either mole or cation percent. It is undefined in the absence of alkali oxides.

R-values are included in the tables above, where appropriate. High positive R-values, especially around 1 or greater, seem undesirable.

While typical embodiments have been set forth for the purpose of illustration, the foregoing description should not be deemed to be a limitation on the scope of the disclosure or the appended claims. Accordingly, various modifications, adaptations, and alternatives may occur to one skilled in the art without departing from the spirit and scope of this disclosure or the appended claims.

1-24. (canceled)

25. A glass article comprising copper selected from the group consisting of Cu ions, metallic copper, colloidal copper, copper nanoparticles, and combinations thereof dispersed throughout the glass and at a surface of the glass; and the glass having antimicrobial properties.

26. The article according to claim 25, wherein the copper is in a reduced state.

27. The article according to claim 26, wherein the reduced copper is at a depth of in the range of from 2 μm to 3 μm from the surface of the glass.

28. The article according to claim 26, having a log reduction ≧1.

29. The article according to claim 25, wherein the glass is a strengthened glass.

30. The article according to claim 25, wherein the glass as batched comprises 0.1 mole %-20 mole % copper.

31. The article according to claim 25, wherein the glass as batched comprises 10 mole %-40 mole % B₂O₃.

32. The article according to claim 25, wherein the glass as batched comprises a B₂O₃/Al₂O₃ ratio greater than 1.

33. The article according to claim 25, wherein the glass as batched has an R-value of less than 1.

34. The article according to claim 25, wherein the glass comprises copper nanoparticles and wherein the nanoparticles are adhered to the surface.

35. The article according to claim 25, wherein the glass as batched comprises in mole percent:
40-85 SiO₂;
10-40 B₂O₃;
0-19 Al₂O₃;
0.1-20 CuO;
0-20 M₂O, wherein M is Li, Na, K, or combinations thereof;
0-25 RO, wherein R is Ca, Sr, Mg, or combinations thereof; and
0-20 ZnO.

36. The article according to claim 35, wherein the glass as batched comprises in mole percent:
40-70 SiO₂;
16-31 B₂O₃;
3-15 Al₂O₃;
5-15 CuO;
0-20 M₂O, wherein M is Li, Na, K, or combinations thereof;
0-25 RO, wherein R is Ca, Sr, Mg, or combinations thereof; and
0-17 ZnO.

37. The article according to claim 35, wherein the glass as batched is phosphorus free.

38. The article according to claim 25, wherein the glass is an aluminoborosilicate or borosilicate glass.

39. The article according to claim 25, wherein the glass consists essentially of SiO₂=47±2 mole %, Al₂O₃=9±1-1.5 mole %, B₂O₃=27±3 mole %, 7-16±1.5 mole % for ZnO, and Cu being 0.5-10±0.2-1.5 mole % as the copper content increases.

40. A method of making a copper-containing glass article having antimicrobial properties, the method comprising:
batching a glass batch comprising in mole percent:
40-85 SiO₂;
10-40 B₂O₃;
1-19 Al₂O₃;
0.1-20 CuO or a selected salt of Cu that is convertible into CuO during melting;
0-20 M₂O, wherein M is Li, Na, K, or combinations thereof;
0-25 RO, wherein R is Ca, Sr, Mg, or combinations thereof; and
0-20 ZnO.
melting the batch to form a melted glass; and
forming the melted glass to form the copper-containing glass article having antimicrobial properties.

41. The method according to claim 40, further comprising heating the article in a reducing atmosphere at an elevated temperature in the range of from 250° C. to 475° C. thereby reducing the copper ions, Cu⁺², in the glass as an oxide or other species, to the metal, Cu⁰.

42. The method according to claim 41, wherein the heating comprises heating the article for a time in the range of from 2 hours to 5 hours.

43. The method according to claim 40, further comprising strengthening the article after the forming.

44. The method according to claim 43, wherein the strengthening comprises ion-exchanging alkali metal ions in the article for alkali metal ions that have a larger ionic radius.
